(19) United States
(12) Patent Application Publication
(10) Pub. No.: US 2022/0186261 A1
(43) Pub. Date: Jun. 16, 2022

(54) Use of Haploid Embryonic Cells to Generate Offspring with Predetermined Genomes

(71) Applicants: THE SEMEX ALLIANCE, Guelph (CA); UNIVERSITÉ DE MONTRÉAL, Montreal (CA)

(72) Inventors: Lawrence Charles Smith, Saint-Hyacinthe (CA); Remi Labrecque, St-Pie (CA); Luis Miguel Aquila Paredes, Saint-Hyacinthe (CA); Jacinthe Therrien, Mont St-Hilaire (CA); Christian Vigneault, St-Pie (CA)

(21) Appl. No.: 17/430,652

(22) PCT Filed: Feb. 19, 2020
(86) PCT No.: PCT/CA2020/050210

§ 371 Date: Aug. 12, 2021

Related U.S. Application Data
(60) Provisional application No. 62/808,106, filed on Feb. 20, 2019.

(51) Int. Cl. C12N 15/877; C12N 5/073; G16B 20/40

(52) U.S. Cl. CPC C12N 15/8771; G16B 10/00; G16B 20/40; C12N 5/0604

(57) ABSTRACT

A method of generating offspring with a precharacterized genome is disclosed. In one embodiment, the method comprises the steps of (a) obtaining ungulate haploid embryonic cells, (b) deriving haploid embryonic outgrowth from the cells of step (a), (c) characterizing the genome of the haploid cells of step (b), and (d) deriving diploid embryos or offspring from the cells of step (c).

### CROSS-REFERENCE TO RELATED APPLICATIONS

This application claims the benefit of U.S. Provisional Patent Application No. 62/808,106, filed Feb. 20, 2019, titled “Use of Haploid Embryonic Cells to Generate Offspring with Predetermined Genomes,” which is incorporated by reference herein in its entirety.

### STATEMENT REGARDING FEDERALLY SPONSORED RESEARCH

N/A

### BACKGROUND

Animal selection has traditionally been performed using phenotypic records and pedigrees in which superior animals are chosen as parents according to an estimated breeding value (EBV). Such traditional strategies have been successful for traits with high heritability, e.g. genetic improvement for milk yield has increased consistently for many decades. However, progeny testing and pedigree information has proven less effective with traits with low heritability. Moreover, an accurate EBV is costly and time consuming to obtain due to the recording and analysis of many individuals and to the long generation intervals in cattle. Indeed, identifying elite dairy sires by EBV relies on a tedious progeny-testing scheme that takes 6-7 years and costs approximately $50,000 per bull tested.

Recent advances in molecular genetics have enabled a novel selection strategy for identifying genetically superior parents by the use of DNA markers associated with quantitative traits (Meuwissen et al. 2001). A key breakthrough in marker-assisted selection came with the sequencing of the whole bovine genome (Bovine HapMap et al. 2009), which has led to the discovery of many thousands of DNA markers in the form of single nucleotide polymorphisms (SNP) associated with production traits and other traits of interest. These novel molecular tools have enabled a dramatic reduction in the cost of genotyping.

A second major breakthrough came with the demonstration that it is possible to make accurate selection decisions when breeding values are predicted from DNA markers alone by calculating genomic breeding values (GEBV).

The implications of achieving accurate GEBV for animals at birth are profound. Potentially, genomic selection can lead to a doubling of the rate of genetic gain through selection and breeding from bulls at 2 years of age rather than 5 years of age or later (Schaeffer 2006). Although more genotyping is needed to increase selection intensity and thereby increase the rates of genetic gain, it is expected that cattle breeding companies can save a large majority of their costs using GEBV instead of the traditional EBV (Hayes et al. 2009). It has now become possible to evaluate the genetic merit of a newborn calf or even a preimplantation stage embryo, provided that a reference population is available, with comparable accuracy for less than $100 (Georges 2014).

Although cattle generation intervals can be dramatically decreased and selection accuracy can be greatly improved by using the genomic approach and selecting genetically superior offspring very early post-fertilization, selection programs are consistently limited by independent assortment and crossing over of parental chromosomes during meiosis, causing uncontrollable genomic variability prior to fertilization.

Meiotic genetic diversity is ensured during two events, crossing over and independent assortment of chromosomes. Crossing over occurs during prophase I of meiosis, and enables homologous pairs of chromosomes to recombine and often exchange chromosome segments. This allows genes from each parent to intermix and create chromosomes with a different genomic complement. Independent chromosome assortment occurs during meiosis II when sister chromatids separate and are randomly distributed to the daughter cells, i.e. gametes. In cattle, independent assortment can yield 230, or 1,073,741,824, unique ways to arrange 30 pairs of chromosomes. To date, all selection strategies are performed post-fertilization when the random combination of paternal and maternal genomes has already occurred. It would be greatly advantageous to eliminate meiotic uncertainty by selecting genetically superior gametes prior to fertilization. Therefore, we propose the present invention, wherein haploid cell lines derived preferably from male and female gametes, or somatic cells, can be obtained and analyzed to select those carrying superior genomic markers prior to fertilization. The most promising haploid cells can then be ‘reconstructed,’ i.e. fertilized, to produce embryos and offspring with predetermined genomes.

Diploid genomes (two sets of chromosomes, one maternal and one paternal) are typical among most living animals, and haploidy (a single set of chromosomes) is generally limited to gametes. Although a diploid genome is thought to increase fitness by masking mutations, it also leads to the accumulation of mutations with time. To counteract long-term degradation of the genome, mammals created adaptations that include genomic imprinting, random monoallelic expression and X chromosome inactivation (Wutz 2014). Haploidy is normally restricted to the post-meiotic stages of germ cells and represents the end point of cell proliferation, which means that physiological haploidy is incompatible with self-renewal.

However, the recent advent of haploid mouse embryonic stem cell (ESC) technologies has drastically changed this situation (Kokubu & Takeda 2014). Recently, some studies have looked at deriving ESCs from mammalian parthenogenetic and androgenetic haploid embryos. These initial studies were focused on mouse ESCs (Elling et al. 2011; Leeb & Wutz 2011; Li et al. 2012; Yang et al. 2012). Similar techniques have been applied to monkey (Yang et al. 2013) and rat haploid ESC derivation (Li et al. 2014).

The original versions of haploid ESC lines (Elling et al. 2011; Leeb et al. 2014) were generated by parthenogenetic activation of unfertilized mouse oocytes with chemicals such as strontium salt or ethanol. These haploid mouse ESCs contain only the maternal set of chromosomes, and show pluripotency as well as self-renewal capabilities. Androgenetic haploid mouse ESC lines containing only the paternal chromosomes have also been generated by removal of the maternal pronucleus from zygotes and by introduction of sperm into enucleated oocytes (Li et al. 2012; Yang et al. 2012). Thus, pluripotency, self-renewal, and haploidy can be incorporated together in a single cell line.

Haploid ESC lines have been shown to function as gametes and support further embryonic development (Li et al. 2012; Yang et al. 2012; Wan et al. 2013; Shuai & Zhou 2014). Metaphase oocytes were ‘fertilized’ with haploid ESC by intracytoplasmic cell injection resulting in the production of fertile pups.

In another experiment sperm were injected into an enucleated oocyte, followed by the activation of the reconstructed embryos by chemical stimulus. Pups were generated, albeit at low efficiencies (Wan et al. 2013).

Although haploid ESC have never been reported in domestic species, diploid embryonic stem-like cells have been isolated previously from in vitro fertilized, nuclear transfer and parthenogenetic diploid embryos (Stice et al. 1996; Betts et al. 2001; Talbot et al. 2005; Wang et al. 2005; Pashaiasl et al. 2010; Jin et al. 2012, Bogliotti et al, 2018). One of the major barriers in deriving genuine ESC lines in ungulate species relates to their tendency of undergoing differentiation to ‘cobblestone-like’ epithelial cells that show either lengthy cell cycles or complete cell cycle arrest after prolonged culture periods (Talbot et al. 2005; Desmarais et al. 2011). Nonetheless, recent studies have indicated that stable primed pluripotent embryonic stem cells can be obtained from bovine IVF blastocyst stage embryos (Bogliotti et al, 2018).

Such tendency to differentiate is further exacerbated when cultures are performed in the absence of feeder-layers. To eliminate the risk cell contaminants from different species, e.g. mitomycin-treated mouse embryonic fibroblasts, different types of extracellular matrix (ECM) such as lamin, gelation or combination of both, have been successfully used to derive bovine ESC lines (Verma et al. 2013). Together, the background research described above provides a base and background for the present invention.

To summarize, the present invention is aimed at reducing meiotic uncertainty from breeding programs, preferably ungulate breeding programs, by determining the genomic value of paternal and/or maternal gametes and screening or selection of those gametes before creation of a diploid embryo.

### SUMMARY OF THE INVENTION

In one embodiment, the present invention is a method of generating mammalian diploid embryos and/or offspring with a pre-characterized genome comprising the steps of (a) obtaining an embryonic haploid cell, (b) deriving haploid outgrowth from the cell of step a, (c) characterizing the genome of the haploid cells of step a or b, and (d) deriving diploid embryos and/or offspring from the cells of steps a, b or c. In a preferred embodiment, the embryonic haploid cell is an ungulate cell. In another preferred embodiment, the cell is a bovine cell.

In one embodiment, the embryonic haploid cell is derived from an ungulate parthenogenetic haploid embryo in vitro cultured. The embryo may be prepared by a method comprising haploid parthenogenetic activation of an ungulate oocyte.

In another embodiment, the haploid cell is derived from an ungulate androgenetic haploid embryo in vitro cultured. The embryo may be prepared by a method comprising removing the ungulate oocyte's genome either before or after fertilization by the intracytoplasmic sperm injection (ICSI) of a single sperm or in vitro fertilization (IVF).

In one embodiment, the haploid outgrowth comprises cellular multiplication to produce a larger number of haploid cells, the method comprising the in vitro culturing of a haploid cell isolated from a preimplantation embryo. The haploid embryonic cell may isolated from a preimplantation embryo at a cleavage, four cells, eight cells, or sixteen cells, or from a morula or blastocyst stage embryo.

In one embodiment, characterizing the genome of the haploid cells comprises performing haploid genomic scoring of the haploid cell. That scoring may include (i) scoring the genomic screening the haploid embryonic-derived cell or (ii) selecting the haploid embryonic-derived cells for the preferred haploid genomic score or selection index. The score may comprise including a weighted combination of one or more single nucleotide polymorphisms. The score may be based on traits selected from the group consisting of production traits (e.g. milk, fat, protein, fat %, protein %, composition of milk protein variants), health traits (e.g. somatic cell score, mastitis resistance, immune response, livability, disease resistance), reproductive traits (e.g. pregnancy rate, conception rate), calving traits (e.g. calving ease, calving to first insemination, stillbirths), conformation traits (e.g. polled trait, udder and teat traits, feet and leg traits, body traits, dimension traits), efficiency traits (e.g. feed efficiency traits, workability, longevity, productive life), novel traits (e.g. robotic milking traits, heat tolerance, activity traits and behavior traits), and composite index traits (e.g. LPI (Life Production Index), and TPI (Total Production Index).

In one embodiment, obtaining the derived diploid embryos and/or offspring with a precharacterized genome may comprise introducing the genomically characterized haploid cell isolated from a haploid preimplantation embryo or outgrowth line into an oocyte.

In another embodiment, the haploid embryonic-derived cell carrying the preferred genomic score is used for maternal embryo reconstruction, the method additionally comprising introducing an ungulate haploid embryonic-derived cell isolated from a parthenogenetic haploid preimplantation embryo or outgrowth line into a fertilized and enucleated ungulate oocyte before paternal pronuclear formation.

In one embodiment, the haploid embryonic-derived cell carrying the preferred genomic score is used for paternal embryo reconstruction, the method additionally comprising introducing a haploid embryonic-derived cell isolated from an androgenetic haploid preimplantation embryo or outgrowth line into a parthenogenetically-activated oocyte before maternal pronuclear formation.

In another embodiment, the haploid embryonic-derived cells carrying the preferred genomic score are used for biparental embryo reconstruction, the method additionally comprising introducing both a haploid embryonic-derived cell isolated from an androgenetic haploid preimplantation embryo or outgrowth line and an ungulate haploid embryonic-derived cell isolated from a parthenogenetic haploid preimplantation embryo or outgrowth line into an oocyte.

In one embodiment, offspring are derived by a method comprising implanting the reconstructed diploid into a recipient host. In another embodiment, the introducing of the haploid cell into the oocyte comprises electrofusion or cell injection using micromanipulation approaches. In preferred embodiments, the haploid embryonic-derived cell is isolated from a preimplantation embryo at the cleavage, four cells, eight cells, sixteen cells, morula, or blastocyst stages, or isolated from a haploid outgrowth.

In one embodiment, the cells of step a, b, or c are characterized by screening for preferred genetic or genomic characteristics. The screening may be selected from the method selected from the group consisting of:
i) genomic selection,
ii) marker-assisted selection,
iii) selection against deleterious haplotypes or alleles, and
iv) selection for favorable haplotypes or alleles.

In one embodiment, the screening or selection of the cells of step (a), (b), or (c) are selected after assessment of homozygosity of the cells and/or their genomic imputation and estimation genomic breeding value of each haploid embryo.

In one embodiment, screening or selection is done to:
i) create a subpopulation of animals enriched for a particular trait, or
ii) create a subpopulation of animals without a particular trait, or
iii) create a subpopulation of animals with a series of favorable traits and/or without a series of unfavorable traits, or
iv) create a subpopulation of animals without one or more deleterious haplotypes, or
v) create a subpopulation of animals with one or more favorable haplotypes.

In one embodiment, screening or selection is done to create breeding animals or breeding lines that contain a unique combination of alleles, haplotypes, or traits, wherein the alleles, haplotypes, or traits are normally appearing at low frequency in the population, wherein the frequency is normally below 50% of the population.

In one embodiment, the genetic or genomic characteristics are selected from the group consisting of production traits (e.g. milk, fat, protein, fat %, protein %, milk protein variant composition), health traits (e.g. somatic cell score, mastitis resistance, immune response, livability, disease resistance), reproductive traits (e.g. pregnancy rate, conception rate), calving traits (e.g. calving ease, calving to first insemination, stillbirths), conformation traits (e.g. polled traits, udder and teat traits, feet and leg traits, body traits, dimension traits), efficiency traits (e.g. feed efficiency traits, workability, longevity, productive life), novel traits (e.g. robotic milking traits, heat tolerance, activity traits and behavior traits), and composite index traits (e.g. LPI (Life Production Index) and TPI (Total Production Index).

### DESCRIPTION OF THE FIGURES

FIG. 1 shows steps (1-4) in the production of haploid androgenetic embryonic cells.

FIG. 2 shows steps (1-3) in the production of haploid parthenogenetic embryonic cells.

FIG. 3 shows images of developmental stages at different days (2-8) of in vitro cultured bovine embryos derived from diploid ICSI controls, and haploid androgenetic and parthenogenetic zygotes.

FIG. 4 shows images and amounts of nuclei present in morula and blastocyst stage embryos derived from diploid ICSI controls, haploid androgenetic (Androgeno) and parthenogenetic (Partheno) zygotes.

FIG. 5 shows outgrowths of bovine (a) androgenetic and (b) parthenogenetic haploid embryos after (at) 10 days of in vitro culture. Nuclear staining of a (c) day-5 and a (d) day-10 haploid embryo outgrowth containing approximately 325 and 1955 cells, respectively.

FIG. 6 shows steps (1-3) in the production of diploid embryos and offspring with predetermined and selected paternal haploid genomes.

FIG. 7 shows steps (1-3) in the production of diploid embryos and offspring with genomically selected maternal haploid genomes.

FIG. 8 shows steps (1-3) in the production of diploid embryos and offspring with predetermined and selected paternal and maternal haploid genomes.

FIG. 9 shows diploid blastocyst stage embryos (day 7) derived from the reconstruction of parthenogenetically activated oocytes with haploid androgenetic embryonic cells.

### DEFINITIONS

The practice of the present invention will employ, unless otherwise indicated, conventional techniques of tissue culture, mammalian embryo culture, molecular biology, cell biology and gamete micromanipulation, which are within the skill of the art. See, e.g., Sambrook, Fritsch and Maniatis (1989) Molecular Cloning: A Laboratory Manual, 2nd edition; F. M. Ausubel, et al. eds. (1987) Current Protocols In Molecular Biology; the series Methods in Enzymology (Academic Press, Inc.): PCR 2: A Practical Approach (1995) (M. J. MacPherson, B. D. Hames and G. R. Taylor eds.); and R. I. Freshney, ed. (1987) Animal Cell Culture.

All numerical designations, e.g., pH, temperature, time, concentration, and molecular weight, including ranges, are approximations which are varied (+) or (−) by increments of 1.0 or 0.1, as appropriate. It is to be understood, although not always explicitly stated that all numerical designations are preceded by the term “about.” It also is to be understood, although not always explicitly stated, that the reagents described herein are merely exemplary and that equivalents of such are known in the art.

As used in the specification and claims, the singular form “a,” “an,” and “the” include plural references unless the context clearly dictates otherwise.

As used herein, “ploidy” is the number of complete sets of chromosomes in a cell, and hence the number of possible alleles for autosomal and sexual genes. Somatic cells, tissues, and individual organisms can be described according to the number of sets of chromosomes present (the “ploidy level”): haploid (1 set), diploid (2 sets), triploid (3 sets), tetraploid (4 sets), pentaploid (5 sets), etc. The generic term “polyploid” is often used to describe cells with three or more chromosome sets.

“Diploid” is meant to indicate a cell, e.g., a gamete or blastomere, having two sets of chromosomes of paternal, maternal or both origins.

“Haploid” is meant to indicate a cell, e.g., a gamete or blastomere having one set of chromosomes of paternal or maternal origin.

“Oocyte activation” indicates wherein a fertilized or unfertilized oocyte, preferably in metaphase II of meiosis, undergoes a process typically including extrusion of the second polar body, exocytosis of cortical granules (CGs), meiotic cell-cycle resumption, pronuclear formation, translation of maternal mRNAs, and meiosis-to-mitosis transition. In the present invention, “oocyte activation” refers to methods whereby an ungulate oocyte containing DNA of paternal and/or maternal origin is induced to develop by natural or artificial fertilization and/or through mechanical, chemical, and/or electrical stimuli into an embryo that has a discernible inner cell mass and trophectoderm. Methods of performing oocyte activation are known in the art. See, for example, Cibelli et al. (2002) Science 295(5556):819 and Vrana et al. (2003) Proc. Natl. Acad. Sci. USA 100(Suppl. 1)11911-6.

“Metaphase II” stage of cell cycle is wherein the DNA content of an oocyte consists of a haploid number of chromosomes with each chromosome represented by two chromatids.

“Anaphase II”/“Telophase II” refers to the transition-phase of the meiosis II characterized by an asymmetrical cell division, bilateral furrowing, and abscission of the polar body, where the chromatids of each chromosome move to opposite poles still joined on the spindle.

“ICSI,” or “intracytoplasmic sperm injection,” refers to the process wherein one or more sperm are injected directly into an egg using micromanipulation approaches.

By “IVF” or “in vitro fertilization” is meant the process of fertilization wherein an oocyte is combined with sperm outside the body, in vitro.

“Enucleation” refers to removing the ungulate oocyte's genome as aided by micromanipulation techniques.

A “control” is an alternative subject or sample used in an experiment for comparison purposes. A control can be “positive” or “negative.” For example, where the purpose of the experiment is to determine a correlation of an altered expression level of a gene with a particular genotype, it is generally preferable to use a positive control (e.g., a sample from a subject, carrying such alteration and exhibiting the desired phenotype), and a negative control (e.g., a subject or a sample from a subject lacking the altered expression or phenotype).

As used herein, the terms “culture media” and “culture medium” are used interchangeably and refer to a liquid substance used to support the growth of cells (e.g., mammalian embryonic cells). Preferably, the culture media as used herein can be a water-based media including a combination of ingredients such as salts, nutrients, minerals, vitamins, amino acids, nucleic acids, proteins such as cytokines, growth factors, and hormones. For example, a culture media can be a synthetic culture media such as, for example, synthetic oviductal fluid media (SOF), modified SOF, KSOM media (MilliporeSigma, Burlington, Mass., USA), modified KSOM, minimum essential media (MEM) (HyClone Thermo Scientific, Waltham, Mass., USA), DMEM/F12 (Life Technologies, Carlsbad, Calif., USA), Neurobasal Medium (Life Technologies, Carlsbad, Calif., USA), KO-DMEM (Life Technologies, Carlsbad, Calif., USA), DMEM/F12 (Life Technologies, Carlsbad, Calif., USA), supplemented with the necessary additives as is further described herein. In some embodiments, the cell culture media can be a mixture of culture media. Preferably, all ingredients included in the culture media of the present disclosure are substantially pure and tissue culture grade.

The term “culturing” refers to the in vitro propagation of cells or organisms on or in media of various kinds. It is understood that the descendants of a cell grown in culture may not be completely identical (i.e., morphologically, genetically, or phenotypically) to the parent cell.

The term “diploid embryo reconstruction” refers to establishing a diploid embryo from a maternal diploid reconstruction process wherein the maternal diploid reconstruction process comprises (a) inserting a mammalian, preferably an ungulate, haploid parthenogenetic cell into a fertilized and enucleated oocyte, (b) establishing a diploid embryo from a paternal diploid reconstruction process, wherein the paternal diploid reconstruction process comprises inserting the haploid androgenetic embryonic cell into a parthenogenetically-activated oocyte, and (c) establishing a diploid embryo from a biparental diploid reconstruction process, wherein the biparental diploid reconstruction process comprises inserting a haploid parthenogenetic and an androgenetic embryonic cell into an enucleated and activated oocyte.

An ungulate is a hoofed animal, e.g., a bovine, an ovine, an equine, a pig, a giraffe, a camel, a deer, a hippopotamus, or a rhinoceros.

### DETAILED DESCRIPTION OF THE INVENTION

The present disclosure is a method of using haploid embryonic cells to characterize haploid genotypes. In a preferred embodiment, the method is used to generate offspring with predetermined genomes or genetic characteristics.

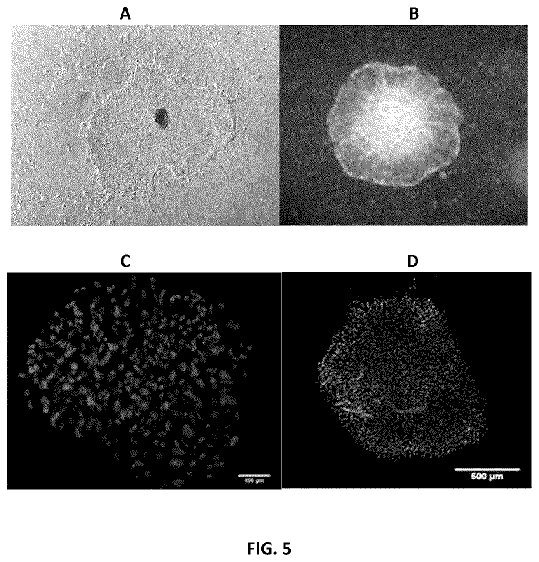

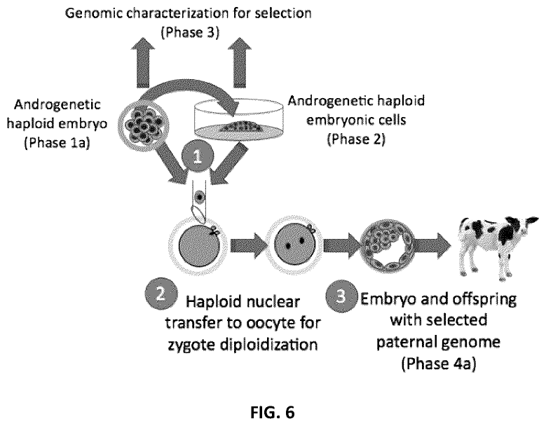

In one embodiment, the method of the present invention comprises four phases, as described below. In brief, one will obtain haploid embryonic cells (either androgenetic or parthenogenetic cells) in phase 1, derive haploid embryonic outgrowth (FIG. 5) and cell lines in phase 2, characterize the genome of the haploid androgenic or parthenogenetic cells in phase 3, and derive diploid embryos/offspring from the androgenetic and/or parthenogenetic embryonic cells in phase 4 (FIGS. 6, 7, and 8).

One preferred goal of the present invention is to obtain preferred genetic characteristics in an embryo or offspring. For example, one may wish to obtain the following characteristics in a bovine embryo: in some embodiments of the invention, we would look at production traits (fat, protein, milk production, milk protein variant composition, e.g. A2A2 milk), health traits (e.g. somatic cell score, mastitis resistance, metabolic disease resistance, reproductive disease resistance, immune response, livability or reduced incidence of various diseases), reproductive traits (e.g. daughter pregnancy rate, sire conception rate), calving traits (e.g. calving ease, calving to first insemination, stillbirths), conformation traits (e.g. udder and teat traits, feet and leg traits, body traits, dimension traits), efficiency traits (e.g. feed efficiency traits, workability, longevity, productive life), novel traits (e.g. polled trait, heat tolerance, robotic milking traits, activity traits and behavior traits), LPI (Life Production Index—more popular in Canada), or TPI (Total Production Index—more popular in USA) Productive Life. In another version of the present invention, one may wish to eliminate undesired traits (e.g. horned trait, various deleterious haplotypes, etc.).

The method of the present invention is useful for any mammalian species (preferably non-human mammals) or, more preferably, any ungulate species. A preferred version of the invention uses commercially important species such as bovine, equine, ovine, caprine, and porcine species. Most preferably, one would use bovine gametes (sperm and oocytes) for the phases. One could also use somatic cell lines that are de-differentiated into pluripotent cells and use the approach described herein to produce haploid cells.

The examples below disclose exemplary or preferred methods of achieving the phases. Of course, other methods may be preferable in some circumstances. For example, the present invention includes the use of fully mature gametes, both male (sperm) or/and female (oocytes).

Another embodiment would include the use of other post-meiotic male (i.e. spermatocytes and spermatids) and female (i.e. second polar bodies) gametes/cells to produce the haploid cells or cell lines. One could also use pre-meiotic cells (i.e. spermatogonia, oogonia or stem cells) and then enable meiotic resumption in vitro to produce the haploid cells or cell lines. Additionally (and as mentioned previously), one could induce meiosis in any somatic cell (stem cell or not).

Phase 1—Derivation of Haploid Embryonic Cells.

Rationale: Multiple haploid embryonic cells are obtained from paternal (sperm) and maternal (oocyte) individual gametes by androgenetic and parthenogenetic development, respectively.

Exemplary Methodology:

Note: the experimental protocol below is based on our actual experimental work with bovine haploid androgenetic (Phase 1a) and parthenogenetic (Phase 1b) embryonic cells, derivation of haploid embryonic outgrowths (Phase 2), genomic screening and selection (Phase 3), and derivation of diploid embryos with characterized parental genomes using haploid embryonic cells (Phase 4). The description of the derivation of offspring below is prophetic. We have offered substitutions and suggestions for variant method steps based on our vision of prophetic embodiments.

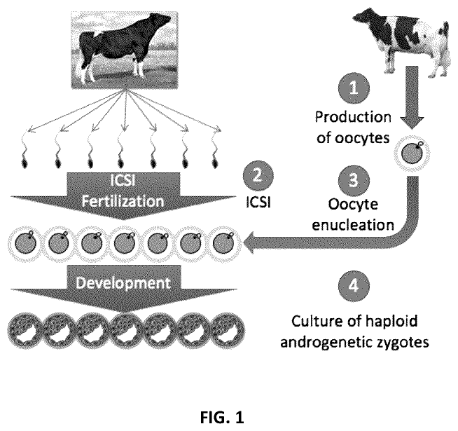

Phase 1a—Derivation of Haploid Androgenetic (Sperm-Derived) Embryonic Cells (FIG. 1).

Step 1. Preparation of in vitro matured (MII) bovine oocytes (similar to Hansen 2013):
Cumulus-oocyte-complexes (COCs) are aspirated from follicles≥2 mm in size;
COCs are washed, placed in maturation medium, and cultured for 20 to 28 h at 38° C. in an atmosphere of 5% CO₂ in air;
After 1- to 3-minute exposure of COCs to medium at 37° C. containing 1 mg/ml of hyaluronidase, oocytes are stripped of cumulus cells mechanically;
Only MII oocytes are selected for further use by the presence of a polar body and homogeneous cytoplasm;

Step 2. Intracytoplasmic bovine sperm injection (ICSI) of oocytes (similar to Matcuit et al, 2006)
Note that we could also use in vitro fertilization (IVF) instead of ICSI to derive fertilized oocytes.
Conventional frozen semen is thawed, preferably in a 37° C. water bath, and centrifuged at 600 g for 5 minutes in 0.6 ml Percoll gradient (45/90%). Note that one could substitute frozen semen with fresh semen or any other post-meiotic male source; Percoll-separated or sperm separated, or not, with other separation protocols; non-sexed or sexed sperm.
A 30 μl sample of sperm is removed from the pellet and centrifuged a second time at 300 g for 2 minutes.
A 1 μl of diluted sperm is placed in a 4 μl drop of 5-10% PVP (polyvinylpyrrolidone).
A sperm, preferably actively moving, is aspirated into a microinjection pipette, immobilized and injected into the cytoplasm of a MII oocyte by traversing the zona pellucida and the plasma membrane.

Step 3. Removal of the chromosomes from ICSI-derived zygotes (oocyte enucleation as described by Bordignon, Smith, 1998). Note that we could also remove the chromosomes from IVF-derived (conventional in vitro fertilization) fertilized zygotes.
At approximately 1 to 5 h after ICSI or sperm penetration (other timing could be used), an oocyte containing a second polar body (telophase II or TII) is selected for enucleation and transferred to a micromanipulation drop containing 1 to 5 μg/ml of cytochalasin B (enucleation medium);
Using an enucleation pipette of approximately 15 μm diameter, the TII chromosomal spindle is aspirated from the ICSI or IVF-derived zygote;
This step enables the production of a haploid androgenetic zygote (1-cell stage embryo carrying only the paternal-derived genome).
Note: Enucleation (step 3) can also be performed before ICSI or IVF (step 2). In that case, oocytes are enucleated by the microsurgical removal of the MII spindle and then fertilized using ICSI (MII enucleation; similar to Bordignon et al 1998).

Step 4. Culture of the androgenetic haploid zygote (similar to Hansen 2013):
Haploid androgenetic zygotes are placed in culture medium in a humidified incubator at 38° C. and allowed to develop for several days. The mentioned culture media is bovine synthetic oviductal fluid (SOF) or modified SOF (e.g., lacking fetal calf serum). Optionally, other culture media similar to or identical to, but not restricted to, KSOM, DMEM, N2B27, TCM-199, or E6 could be substituted.
Assessment of haploid development is performed at different time points to determine cleavage (2- to 8-cell), morula (>32 cells) and blastocyst (approximately 120 cells) rates at 2, 6, and 7 days, respectively. Note that 2 and 3 can be exchanged in FIG. 1.

Phase 1b—Derivation of Haploid Parthenogenetic (Oocyte-Derived) Embryonic Cells.

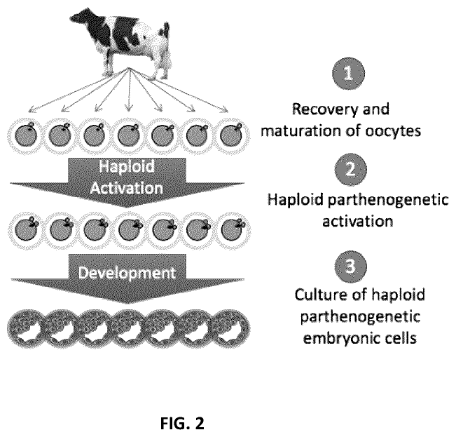

Methodology (FIG. 2):

Step 1. Preparation of in vitro matured (MII) oocytes (as described in Phase 1a):
Cumulus-oocyte-complexes (COCs) are aspirated follicles≥2 mm in size;
COCs are washed, placed in maturation medium, and cultured for 20 to 28 h at 37° C. in an atmosphere of 5% CO₂ in air;
After 1- to 3-minute exposure of COCs to medium at 37° C. containing 1 mg/ml of hyaluronidase, oocytes are stripped of cumulus cells mechanically;
Only MII oocytes are selected for further use by the presence of a polar body and homogeneous cytoplasm;

Step 2. Haploid parthenogenetic activation (similar to Alberio et al, 2001 and Suvá et al, 2019):
Denuded MII oocytes are exposed for 4 to 5 minutes to 5 μm ionomycin (calcium salt), then to 10 μg/ml of cycloheximide for 3 to 7 hours. Note that alternatively, other artificial or natural (not chemical) protocols of activating oocytes can be substituted, including removing the sperm at any time after fertilization and before pronuclear syngamy, or the single or combinate use of ethanol, strontium, 6-DAMP, Roscovitine, PD0325901, and/or mammalian phospholipase Czeta injection (PLCzeta).
Oocytes that expelled a second polar body (putative haploid zygotes) are selected for culture.

Step 3. Culture of the parthenogenetic haploid zygote (as above)
Haploid parthenogenetic zygotes are placed in culture medium in an incubator and are allowed to develop for several days. The mentioned culture media is modified bovine SOF (e.g. lacking fetal calf serum). Optionally, other culture media identical or similar to, but not restricted to, KSOM, DMEM, N2B27, TCM-199, or E6 could be substituted.
Assessment of haploid development is performed at different time points to determine if embryos progress throughout embryo development; examples of time-points are cleavage stage (2- to 8-cell), morula stage (>32 cells) and blastocyst stage (approximately 120 cells) rates at 2, 6, and 7 days of culture, respectively;

Results:

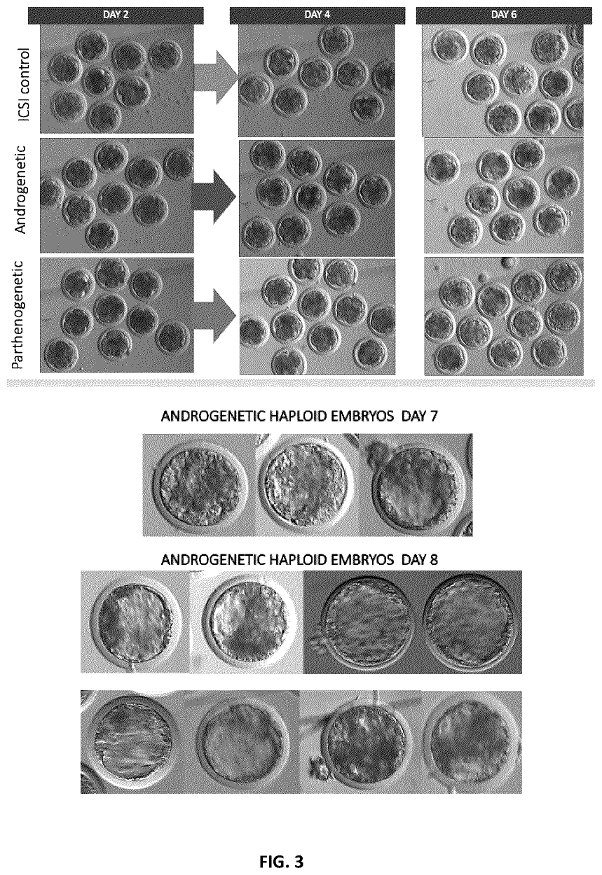

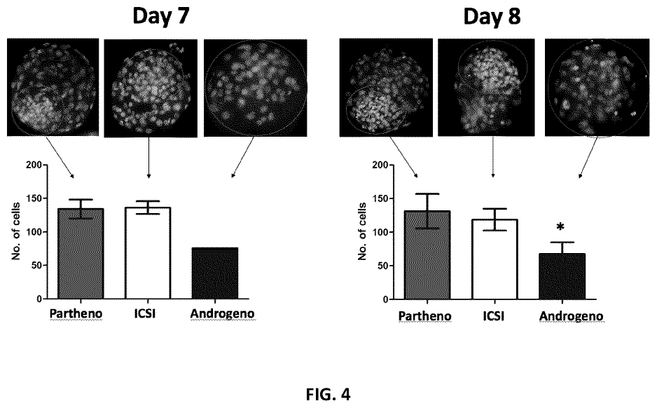

The results described below demonstrate that ungulate (specifically bovine) haploid embryos containing multiple embryonic cells can be obtained by the in vitro culture of both androgenetic and parthenogenetic zygotes (Table 1 and FIG. 3). These results also indicate that embryos of androgenetic origin can be obtained from both X-carrying (haplo-X) and Y-carrying (haplo-Y) sperm (Table 1). These results also indicate that embryos derived after 7 and 8 days of parthenogenetic and androgenetic development contain, on average, over 100 and over 50 nuclei, respectively (FIG. 4). Together, these results clearly demonstrate that multiple ungulate haploid cells can be obtained from single gametes (sperm and oocytes) using the methodological procedures described in Phases 1a and 1b.

TABLE 1
Development in vitro of oocytes fertilized by IVF and ICSI (controls), and
haploid androgenetic and parthenogenetic embryos prepared by different approaches.

| | Fertilized | Cleaved | | Morulas | | Blastocyst | |
|---|---|---|---|---|---|---|---|
| Group | Oocytes | 48 hpi | % | (day 6) | % | (day 7-8) | % |
| IVF (diploid) | 164 | 148 | 90% | 68 | 41% | 61 | 37% |
| ICSI X-carrying (diploid) | 206 | 149 | 72% | 67 | 33% | 55 | 27% |
| ICSI Y-carrying (diploid) | 108 | 82 | 76% | 34 | 31% | 33 | 31% |
| Parthenogenetic haploid | 129 | 79 | 61% | 28 | 22% | 23 | 18% |
| ICSI-Androgenetic haplo-X | 296 | 217 | 73% | 29 | 10% | 8 | 3% |
| ICSI-Androgenetic haplo-Y | 146 | 108 | 74% | 3 | 2% | 0 | N/A |
| IVF Androgenetic haplo-X | 21 | 20 | 95% | 2 | 2% | 0 | 0% |

Phase 2—Derivation of Haploid Embryonic Outgrowth and Cell Lines (FIG. 5). There is the possibility of performing Phase 3 before Phase 2. Moreover, one could skip Phase 2 and go directly from Phase 1 to Phase 3.

Rationale: Haploid embryonic cell lines derived from multicellular ungulate androgenetic and parthenogenetic embryos can be cultured in vitro for prolonged periods to derive an even larger number of haploid embryonic cells.

Methodology:
The zona pellucida of haploid androgenetic and parthenogenetic embryos at the cleavage, morula and/or blastocyst stage is removed by either mechanical (i.e., zona cutting and/or using a fine bore pipette) or enzymatic digestion (i.e., protease or pronase exposure for 1-5 minutes).
Cells from embryos are isolated mechanically and can vary depending on the stage of the embryo (i.e. dissection of inner cell mass from trophoblast layer for blastocyst stage embryos), enzymatically (pronase or protease), or by immunosurgery (i.e., one could substitute exposure to bovine IGG followed by serum complement) or other cell separation techniques.
Using mitomycin-inactivated murine embryonic fibroblasts (MEF) as feeder layers, ungulate haploid embryonic cells are fixed mechanically to the dish coated with 0.1% gelatin in a medium composed of Knockout DMEM with Glutamax supplemented with 10% Knockout or Hyclone serum or alternatively bovine serum albumin (BSA), 0.1 mM β-Mercaptoethanol, 0.1 mM nonessential amino acids, 100 IU/mL penicillin, 0.05 mg/mL streptomycin, 20 ng/mL LIF, and 10 ng/mL bFGF. Note that one could substitute with other feeder cell types, medium, serum source, synthetic molecules as organic matrix such as Matrigel or vitronectin, or nothing at all.
After embryonic cells have been cultured for 6 to 10 days, embryonic haploid colonies can be used for genomic screening, genomic scoring, be frozen, used for diploidization steps and/or passaged mechanically or enzymatically by dissecting with a pipette with or without treatment with trypsin or triple-express medium, washed, and passaged into previously mentioned culture medium with MEF.

Results:

Results in Table 2 and FIG. 5 indicate that, in order to obtain a large number of cells, haploid parthenogenetic and androgenetic embryos can be multiplied in vitro for a number of days.

TABLE 2
Establishment of embryo stem-like outgrowth and early stage cell
lineages in diploid (controls) and haploid parthenogenetic embryos.

| | | | | Out- | | | |
|---|---|---|---|---|---|---|---|
| | Embryo | At- | | growth | | Passage 1 | |
| Group | number | tached | (%) | (Day 7) | (%) | (Day 14) | (%) |
| Diploid | 29 | 11 | 38% | 10 | 34% | 6 | 21% |
| ICSI | | | | | | | |
| Diploid | 7 | 2 | 29% | 2 | 29% | 2 | 29% |
| Parthe- | | | | | | | |
| nogenetic | | | | | | | |
| Haploid | 9 | 5 | 56% | 5 | 56% | 4 | 44% |
| Parthe- | | | | | | | |
| nogenetic | | | | | | | |
| Haploid | 4 | 1 | 25% | 1 | 25% | Ongoing | N/A |
| Andro- | | | | | | (prophetic) | |
| genetic | | | | | | | |

Phase 3—Genomic Characterization of Haploid Androgenetic and Parthenogenetic Embryonic Cells.

Rationale: Genomic characterization can be obtained from a small sample of androgenetic and parthenogenetic haploid embryonic cells. In a preferable method of the present invention, the target cells are characterized by screening, scoring, or selecting for preferred genetic or genomic characteristic. “Screening” typically refers to detecting (preferably through genotyping) the presence or absence of certain commercially valuable traits. “Scoring” typically refers to screening for multiple traits at once, then creating a score that is made up of a series of traits that are equally or differentially weighted according to their relative value. Selection then refers to identifying a subpopulation of animals in the population with the highest scores. “Selection” can also refer to a genomic value made up of hundreds or thousands of genomic markers that span the entire genome and constitute a genomic prediction of the breeding value or transmitting ability of an individual. A subpopulation of animals is then selected on the basis of their genomic breeding values or genomic transmitting ability.

In one embodiment, the cells are scored wherein characterizing the genome of the haploid cells comprises performing haploid genomic scoring of the haploid cell. The haploid genomic scoring may comprise (i) genomically screening the haploid embryonic-derived cell or (ii) selecting the haploid embryonic-derived cells for the preferred haploid genomic score.

In another embodiment, the haploid genomic score comprises including a weighted combination of one or more of one or more single nucleotide polymorphisms.

In one embodiment, the cells are screened via methods selected from the group consisting of:
i) genomic selection,
ii) marker-assisted selection,
iii) selection against deleterious haplotypes or alleles, and
iv) selection for favorable haplotypes or alleles.

In another version of the method, the screening or selection of the cells is after assessment of homozygosity of the cells and/or their genomic imputation and estimation genomic breeding value of each haploid embryo.

In one version of the method, screening, scoring, or selection is done to:
i) create a subpopulation of animals enriched for a particular trait, or
ii) create a subpopulation of animals without a particular trait, or
iii) create a subpopulation of animals with a series of favorable traits and/or without a series of unfavorable traits, or
iv) create a subpopulation of animals without one or more deleterious haplotypes, or
v) create breeding animals or breeding lines that contain a unique combination of alleles, haplotypes or traits, wherein the alleles, haplotypes or traits are normally appearing at a “low” frequency, typically below 50% of the population but present at least 0.1% or at least 0.01%.

In another preferred version of the invention, the genetic or genomic characteristics are selected from the group consisting of production traits (e.g. milk, fat, protein, fat %, protein %, milk protein variant composition), health traits (e.g. somatic cell score, mastitis resistance, immune response, livability, disease resistance), reproductive traits (e.g. pregnancy rate, conception rate), calving traits (e.g. calving ease, calving to first insemination, stillbirths), conformation traits (e.g. polled traits, udder and teat traits, feet and leg traits, body traits, dimension traits), efficiency traits (e.g. feed efficiency traits, workability, longevity, productive life), novel traits (e.g. robotic milking traits, heat tolerance, activity traits and behavior traits), and composite index traits (e.g. LPI (Life Production Index), and TPI (Total Production Index).

Typical Methodology:
Haploid androgenetic or parthenogenetic embryos at any stage, but preferably at the morula or blastocyst stages, are preferably biopsied to remove a few cells (approximately 5 to 10) and the remaining embryo is either used fresh or cryopreserved for future use in establishing a cell line and/or future diploidizations (Phase 4). A variant of this step would be that one could establish a cell line with the entire embryo, then take a subset of the cells to perform the genomic evaluation and decide to either keep the cell line alive while waiting for the genomic results or freeze the cell line and thaw it later once one has the genetic information.
DNA is extracted from the biopsied haploid embryonic cells or the established cell line and then amplified to obtain sufficient DNA for genomic analysis. If not enough DNA is collected, DNA will be amplified using a DNA amplification protocol adapted for samples that have small quantities of DNA.
Assessment of homozygosity of the haploid cells lines and their genomic imputations (Sargolzaei, M., J. P. Chesnais and F. S. Schenkel. 2014. A new approach for efficient genotype imputation using information from relatives. BMC Genomics, 15:478 (DOI: 10.1186/1471-2164-15-478)) is performed to obtain the genomic breeding value from each haploid embryo and/or cell. As explained in the review entitled ‘Genomic selection in dairy cattle: Progress and challenges,’ Dr. Hayes, et al, describe genomic selection as, “Genomic selection refers to selection decisions based on genomic breeding values (GEBV). The GEBV are calculated as the sum of the effects of dense genetic markers, or haplotypes of these markers, across the entire genome, thereby potentially capturing all the quantitative trait loci (QTL) that contribute to variation in a trait.” (J Hayes, P J Bowman, A. J. Chamberlain, and M E Goddard, 2006 Journal of Dairy Science 92:433-443, doi:10.3168/jds.2008-1646). A variant of this method is to obtain the genomic breeding value from each haploid embryo without genomic imputation. In other words, with sufficient density of marker genotypes, no markers would need to be deduced from neighboring markers and implied pedigree based on genomic relationships.

Results:
Biopsies were obtained from 6 haploid androgenetic embryos (3 blastocysts and 3 morulas);
DNA extraction, amplification, and genomic analysis were achieved from all 6 embryos, indicating that the evaluation of genomic content can be achieved from individual haploid embryonic cells;
All samples produced homozygote DNA and had paternal confirmation, indicating that they originated from haploid cells from the animal from which the gametes were obtained.
Together, these results confirm that haploid cells obtained from individual gametes can be genomically analyzed to enable the selection or scoring of gametes carrying specific genomes or genes before fertilization.

Phase 4—Diploidization: Derivation of Ungulate Diploid Embryos and Offspring from Androgenetic and/or Parthenogenetic Embryonic Cells.

Rationale: Diploid embryos and viable offspring can be obtained from genomically characterized haploid androgenetic and/or parthenogenetic embryonic cells.

Phase 4a—Derivation of Diploid Embryos and Offspring from Androgenetic Haploid Embryonic Cells (Paternal Embryo Reconstruction).

Methodology (see FIG. 6):

Step 1. Genomically characterized haploid androgenetic cells (Phases 1a or 2, and 3) are dissociated to obtain individualized cells;
When using morula stage or younger haploid embryos (Phase 1), remove the zona pellucida mechanically or enzymatically and incubate the zona-free embryos for 20 minutes in Ca++ and Mg++-free medium with EDTA (disaggregation medium). If one were to use the blastocyst stage embryos, one could use different methods to isolate the inner cell mass. For example, one could use immunosurgery, mechanical microdissection, etc. The haploid embryonic cells could also be derived from cultured embryonic outgrowths or stem cell lines.
After 20 h exposure to disaggregation medium, embryos are placed in disaggregation medium containing 1 to 5 μg/ml of cytochalasin B and, using the P200 or the holding pipette, aspirated and expelled until thorough disaggregation into individual embryonic cells is achieved.
Cells are placed into a drop of media containing (or not) 1 to 5 μg/ml of cytochalasin B and cycloheximide (or an equivalent) in the micromanipulation dish.

Step 2. Haploid androgenetic cells are introduced into the cytoplasm of TII oocytes (with a second polar body) after parthenogenetic activation and before maternal pronuclear formation. Note: Haploid nuclear introduction can be performed by microinjection (as in ICSI) or by fusion of the plasma membrane of the haploid cell to the oocyte. Alternatively, one could use MII oocytes.

Step 3. After the introduction of haploid androgenetic nuclei, reconstructed diploidized zygotes carrying a genomically characterized paternal genome are cultured in vitro (as previously described above) for 7 days. Reconstructed embryos of good quality are either transferred fresh or frozen (to be transferred at a later date) using conventional embryo transfer protocols as described in George E. Seidel, Jr. and Sarah Moore Seidel's Training manual for embryo transfer in cattle, FAO Animal Production and Health, Paper 77.

Phase 4b—Derivation of Diploid Embryos and Offspring from Parthenogenetic Haploid Embryonic Cells (Maternal Embryo Reconstruction).

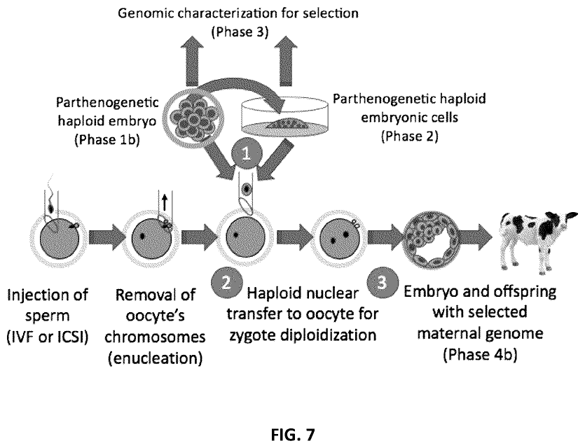

Methodology (see FIG. 7):

Step 1. Genomically characterized haploid parthenogenetic cells (Phases 1b or 2, and 3) are dissociated to obtain individualized cells;
When using morula stage or younger haploid embryos (Phase 1), remove the zona pellucida mechanically or enzymatically and incubate the zona-free embryos for 20 minutes in Ca++ and Mg++-free medium with EDTA (disaggregation medium).
After 20 h exposure to disaggregation medium, embryos are placed in disaggregation medium containing 1 to 5 μg/ml of cytochalasin B and, using the P200 or the holding pipette, aspirated and expelled until thorough disaggregation into individual embryonic cells is achieved.
Cells are placed into a drop of medium containing (or not) 1 to 5 μg/ml of cytochalasin B in the micromanipulation dish.

Step 2. Disaggregated individualized haploid parthenogenetic cells are introduced into the cytoplasm of an enucleated zygote at approximately 2 to 10 h after fertilization and/or sperm penetration in the oocyte (ICSI or IVF). Note: Haploid nuclear introduction can be performed by microinjection (as in ICSI) or by fusion of the plasma membrane of the haploid cell to the oocyte.

Step 3. After the introduction of parthenogenetic haploid nuclei, reconstructed diploidized zygotes carrying a genomically characterized maternal genome will be cultured in vitro (as previously described above) for 7 days;
Reconstructed embryos are transferred to the reproductive tracts of recipient females (surrogates) to obtain gestations and viable offspring. (Applicants have successfully reconstructed the embryo using an androgenetic haploid embryonic cell and a parthenogenetic haploid embryonic cell and created a diploid embryo. Three of these embryos have been transferred. The pregnancies are ongoing, so there are no progeny yet. The description of the progeny below is prophetic.)

Phase 4c—Derivation of Diploid Embryos and Offspring from Androgenetic and Parthenogenetic Haploid Embryonic Cells (Biparental Embryo Reconstruction).

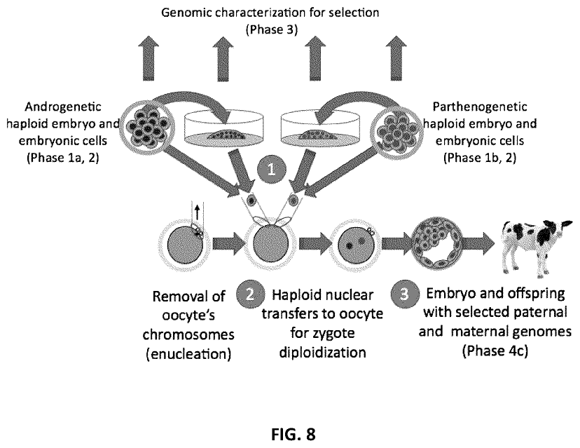

Methodology (see FIG. 8):

Step 1. Genomically characterized haploid androgenetic and parthenogenetic cells (Phases 1a-b, 2a-b, and 3) will be dissociated to obtain individualized cells, most likely by the following method:
When using morula stage haploid androgenetic and parthenogenetic embryos (Phase 1a-b), remove the zona pellucida mechanically or enzymatically, and incubate the zona-free embryos for 20 minutes in Ca++ and Mg++-free medium with EDTA (disaggregation medium).
After exposure to disaggregation medium, embryos are placed in disaggregation medium containing 1 to 5 μg/ml of cytochalasin B and aspirated and expelled until thorough disaggregation into individual embryonic cells is achieved.
Cells are placed into a drop disaggregation medium containing 1 to 5 μg/ml cytochalasin B in the micromanipulation dish.

Step 2. Disaggregated individualized haploid androgenetic and parthenogenetic cells are introduced into the cytoplasm of an oocyte enucleated at TII (with a second polar body) between 2 to 15 h after parthenogenetic activation, most likely by the following method. Note: Haploid nuclear introduction can be performed by microinjection (as in ICSI) or by fusion of the plasma membrane of the haploid cell to the oocyte.

Step 3. After the introduction of both haploid nuclei, reconstructed diploidized zygotes carrying a genomically characterized paternal and maternal genome are cultured in vitro (as previously described in sections [0030] and [0035]) for 7 days;
Reconstructed embryos of good quality are transferred fresh or frozen to synchronized recipients to obtained gestations and viable offspring.

Results:

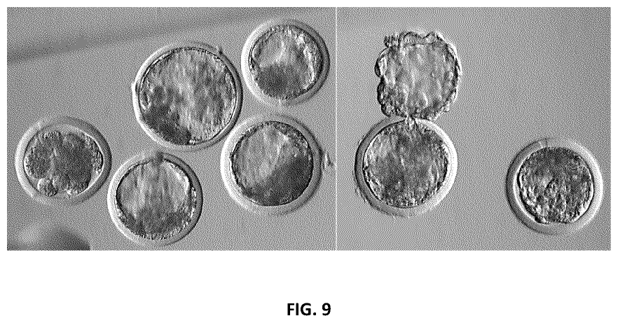

These results demonstrate that bovine diploid embryos produced by reconstructing of oocytes using haploid embryonic cells will cleave at a rate similar to fertilized controls and develop to the blastocyst stage in vitro (Table 3 and FIG. 9).

TABLE 3
Development in vitro of oocytes fertilized by ICSI (controls), and reconstructed
with haploid androgenetic and parthenogenetic.

| | | Cleaved | | Morulae | | Blastocyst | |
|---|---|---|---|---|---|---|---|
| Group | Oocytes | (Day 2) | % | (Day 6) | % | (day 7) | % |
| ICSI (control) | 206 | 149 | 72% | 27 | 33% | 55 | 27% |
| Maternal reconstruction | 23 | 28 | 78% | 5 | 22% | 2 | 22% |
| Paternal reconstruction (X-carrying) | 45 | 33 | 73% | 7 | 16% | 7 | 16% |
| Parental reconstruction (Y-carrying) | 27 | 24 | 89% | 7 | 26% | 7 | 26% |

One may wish to clone or multiply the reconstructed diploid embryo to make multiple copies of the embryo. For example, we have used the ECNT protocols reported in the literature, which includes blastomere separation from bovine embryos developed by Dr. Steen Willadsen (Willadesen, S. M. (1989) Cloning of sheep and cow embryos. Genome 31, 956-962) and production of nuclei from cultured inner cell mass cells from bovine embryos by Drs. Sims and First (Sims, M., & First, N. L. (1994) Production of calves by transfer of nuclei from cultured inner cell mass cells. Proc Natl Acad Sci USA, 91(13), 6143-6147).

### REFERENCES

Alberto R., Zakhartchenko V., Motlik J., Wolf E. (2001) Mammalian oocyte activation: lessons from the sperm and implications for nuclear transfer. Int J Dev Biol 45: 797-809.
Bordignon V., Smith L. C. (1998) Telophase enucleation: an improved method to prepare recipient cytoplasts for use in bovine nuclear transfer. Mol Reprod Dev. 49:29-36.
Hansen P. J. (2013) In Vitro Maturation and Embryo Production in Cattle, University of Florida (http://animal.ifas.ufl.edu/hansen/ivf_docs/University%20of%20Florida%20Bovine%20IVP%20Manual%20ver%2010.16.2013.pdf);
Malcuit C., Maserati M., Takahashi Y., Page R., Fissore R. A. (2006) Intracytoplasmic sperm injection in the bovine induces abnormal [Ca2+]i responses and oocyte activation. Reprod Fert Dev 18: 39-51.
Suvá M., Canel N. G., Salamone D. F. (2019) Effect of single and combined treatments with MPF or MAPK inhibitors on parthenogenetic haploid activation of bovine oocytes. Reprod Biol. 2019 December; 19(4):386-393.
Meuwissen T. H., Hayes B. J. & Goddard M. E. (2001) Prediction of total genetic value using genome-wide dense marker maps. Genetics 157, 1819-29.
Bovine HapMap C., Gibbs R. A., Taylor J. F., Van Tassell C. P., Barendse W., Eversole K. A., Gill C. A., Green R. D., Hamernik D. L., Kappes S. M., Lien S., Matukumalli L. K., McEwan J. C., Nazareth L. V., Schnabel R. D., Weinstock G. M., Wheeler D. A., Ajmone-Marsan P., Boettcher P. J., Caetano A. R., Garcia J. F., Hanotte O., Mariani P., Skow L. C., Sonstegard T. S., Williams J. L., Diallo B., Hailemariam L., Martinez M. L., Morris C. A., Silva L. O., Spelman R. J., Mulatu W., Zhao K., Abbey C. A., Agaba M., Araujo F. R., Bunch R. J., Burton J., Gorni C., Olivier H., Harrison B. E., Luff B., Machado M. A., Mwakaya J., Plastow G., Sim W., Smith T., Thomas M. B., Valentini A., Williams P., Womack J., Woolliams J. A., Liu Y., Qin X., Worley K. C., Gao C., Jiang H., Moore S. S., Ren Y., Song X. Z., Bustamante C. D., Hernandez R. D., Muzny D. M., Patil S., San Lucas A., Fu Q., Kent M. P., Vega R., Matukumalli A., McWilliam S., Sclep G., Bryc K., Choi J., Gao H., Grefenstette J. J., Murdoch B., Stella A., Villa-Angulo R., Wright M., Aerts J., Jann O., Negrini R., Goddard M. E., Hayes B. J., Bradley D. G., Barbosa da Silva M., Lau L. P., Liu G. E., Lynn D. J., Panzitta F. & Dodds K. G. (2009) Genome-wide survey of SNP variation uncovers the genetic structure of cattle breeds. Science 324, 528-32
Hayes B. J., Bowman P. J., Chamberlain A. J. & Goddard M. E. (2009) Invited review: Genomic selection in dairy cattle: progress and challenges. J Dairy Sci 92, 433-43.
Georges M. (2014) Towards sequence-based genomic selection of cattle. Nat Genet 46, 807-9.
Wutz A. (2014) Haploid animal cells. Development 141, 1423-6.
Kokubu C. & Takeda J. (2014) When half is better than the whole: advances in haploid embryonic stem cell technology. Cell Stem Cell 14, 265-7. Elling U., Taubenschmid J., Wirnsberger G., O'Malley R., Demers S. P., Vanhaelen Q., Shukalyuk A. I., Schmauss G., Schramek D., Schnuetgen F., von Melchner H., Ecker J. R., Stanford W. L., Zuber J., Stark A. & Penninger J. M. (2011) Forward and reverse genetics through derivation of haploid mouse embryonic stem cells. Cell Stem Cell 9, 563-74.
Leeb M. & Wutz A. (2011) Derivation of haploid embryonic stem cells from mouse embryos. Nature 479, 131-4.
Li W., Shuai L., Wan H., Dong M., Wang M., Sang L., Feng C., Luo G. Z., Li T., Li X., Wang L., Zheng Q. Y., Sheng C., Wu H. J., Liu Z., Liu L., Wang L., Wang X. J., Zhao X. Y. & Zhou Q. (2012) Androgenetic haploid embryonic stem cells produce live transgenic mice. Nature 490, 407-11.
Yang H., Shi L., Wang B. A., Liang D., Zhong C., Liu W., Nie Y., Liu J., Zhao J., Gao X., Li D., Xu G. L. & Li J. (2012) Generation of genetically modified mice by oocyte injection of androgenetic haploid embryonic stem cells. Cell 149, 605-17.
Yang H., Liu Z., Ma Y., Zhong C., Yin Q., Zhou C., Shi L., Cai Y., Zhao H., Wang H., Tang F., Wang Y., Zhang C., Liu X. Y., Lai D., Jin Y., Sun Q. & Li J. (2013) Generation of haploid embryonic stem cells from Macaca fascicularis monkey parthenotes. Cell Res 23, 1187-200.
Li W., Li X., Li T., Jiang M. G., Wan H., Luo G. Z., Feng C., Cui X., Teng F., Yuan Y., Zhou Q., Gu Q., Shuai L., Sha J., Xiao Y., Wang L., Liu Z., Wang X. J., Zhao X. Y. & Zhou Q. (2014) Genetic modification and screening in rat using haploid embryonic stem cells. Cell Stem Cell 14, 404-14.
Leeb M., Dietmann S., Paramor M., Niwa H. & Smith A. (2014) Genetic exploration of the exit from self-renewal using haploid embryonic stem cells. Cell Stem Cell 14, 385-93.
Wan H., He Z., Dong M., Gu T., Luo G. Z., Teng F., Xia B., Li W., Feng C., Li X., Li T., Shuai L., Fu R., Wang L., Wang X. J., Zhao X. Y. & Zhou Q. (2013) Parthenogenetic haploid embryonic stem cells produce fertile mice. Cell Res 23, 1330-3.
Shuai L. & Zhou Q. (2014) Haploid embryonic stem cells serve as a new tool for mammalian genetic study. Stem Cell Res Ther 5, 20.
Stice S. L., Strelchenko N. S., Keefer C. L. & Matthews L. (1996) Pluripotent bovine embryonic cell lines direct embryonic development following nuclear transfer. Biol Reprod 54, 100-10.
Betts D., Bordignon V., Hill J., Winger Q., Westhusin M., Smith L. & King W. (2001) Reprogramming of telomerase activity and rebuilding of telomere length in cloned cattle. Proc Natl Acad Sci USA 98, 1077-82.
Talbot N. C., Caperna T. J., Powell A. M., Ealy A. D., Blomberg L. A. & Garrett W. M. (2005) Isolation and characterization of a bovine visceral endoderm cell line derived from a parthenogenetic blastocyst. In Vitro Cell Dev Biol Anim 41, 130-41.
Wang L., Duan E., Sung L. Y., Jeong B. S., Yang X. & Tian X. C. (2005) Generation and characterization of pluripotent stem cells from cloned bovine embryos. Biol Reprod 73, 149-55.
Pashaiasl M., Khodadadi K., Holland M. K. & Verma P. J. (2010) The efficient generation of cell lines from bovine parthenotes. Cell Reprogram 12, 571-9.
Jin M., Wu A., Dorzhin S., Yue Q., Ma Y. & Liu D. (2012) Culture conditions for bovine embryonic stem cell-like cells isolated from blastocysts after external fertilization. Cytotechnology 64, 379-89.
Bogliotti Y. S., Wu J., Vilarino M., Okamura D., Soto D. A., Zhong C., Sakurai M., Sampaio R. V., Suzuki K., Izpisua Belmonte J. C. & Ross P. J. (2018) Efficient derivation of stable primed pluripotent embryonic stem cells from bovine blastocysts. Proc Natl Acad Sci USA 115, 2090-5.
Desmarais J. A., Demers S. P., Suzuki J., Jr., Laflamme S., Vincent P., Laverty S. & Smith L. C. (2011) Trophoblast stem cell marker gene expression in inner cell mass-derived cells from parthenogenetic equine embryos. Reproduction 141, 321-32.
Verma V., Huang B., Kallingappa P. K. & Oback B. (2013) Dual kinase inhibition promotes pluripotency in finite bovine embryonic cell lines. Stem Cells Dev 22, 1728-42.

1. A method of generating mammalian diploid embryos and/or offspring with a pre-characterized genome comprising the steps of:
a) obtaining an embryonic haploid cell,
b) deriving haploid outgrowth from the cell of step (a),
c) characterizing the genome of the haploid cells of step (a) or (b), and
d) deriving diploid embryos and/or offspring from the cells of steps (a), (b) or (c).

2. The method of claim 1, wherein the embryonic haploid cell is an ungulate cell.

3. The method of claim 2, wherein the cell is a bovine cell.

4. The method of claim 2, wherein the embryonic haploid cell is derived from an ungulate parthenogenetic haploid embryo in vitro cultured.

5. The method of claim 4, wherein the ungulate parthenogenetic haploid embryo has been prepared by a method comprising haploid parthenogenetic activation of an ungulate oocyte.

6. The method of claim 2, wherein the haploid cell is derived from an ungulate androgenetic haploid embryo in vitro cultured.

7. The method of claim 6, wherein the ungulate androgenetic haploid embryo has been prepared by a method comprising removing the ungulate oocyte's genome either before or after fertilization by the intracytoplasmic sperm injection (ICSI) of a single sperm or in vitro fertilization (IVF).

8. The method of claim 7, wherein the effective timing for removing the ungulate oocyte's genome is between 10 to 1 hours before or 1 to 10 hours after fertilization.

9. The method of claim 6, wherein the embryo is a bovine embryo.

10. (canceled)

11. The method of claim 1, wherein deriving haploid outgrowth comprises cellular multiplication to produce a larger number of haploid cells, the method comprising the in vitro culturing of a haploid cell isolated from a preimplantation embryo.

12. The method of claim 10, wherein the haploid embryonic cell is isolated from a preimplantation embryo at a cleavage, four cells, eight cells, or sixteen cells, or from a morula or blastocyst stage embryo.

13. The method of claim 11, wherein the embryo is a bovine embryo.

14. The method of claim 1, wherein characterizing the genome of the haploid cells comprises performing haploid genomic scoring of the haploid cells to obtain a haploid genomic score, wherein the haploid genomic score comprises a weighted combination of one or more single nucleotide polymorphisms.

15.-17. (canceled)

18. The method of claim 1, wherein deriving diploid embryos and/or offspring with a precharacterized genome comprising introducing the genomically characterized haploid cell isolated from a haploid preimplantation embryo or outgrowth line into an oocyte.

19. The method of claim 18, wherein the haploid embryonic-derived cell carrying the preferred genomic score is used for maternal embryo reconstruction, the method additionally comprising introducing a haploid embryonic-derived cell isolated from a parthenogenetic haploid preimplantation embryo or outgrowth line into a fertilized and enucleated oocyte before paternal pronuclear formation.

20. The method of claim 18, wherein the haploid embryonic-derived cell carrying the preferred genomic score is used for paternal embryo reconstruction, the method additionally comprising: introducing a haploid embryonic-derived cell isolated from an androgenetic haploid preimplantation embryo or outgrowth line into a parthenogenetically activated oocyte before maternal pronuclear formation.

21. The method of claim 18, wherein haploid embryonic-derived cells carrying the preferred genomic score are used for biparental embryo reconstruction, the method additionally comprising introducing both a haploid embryonic-derived cell isolated from an androgenetic haploid preimplantation embryo or outgrowth line and a haploid embryonic-derived cell isolated from a parthenogenetic haploid preimplantation embryo or outgrowth line into an oocyte.

22. The method of claim 18, wherein the embryo is bovine.

23-32. (canceled)

33. The method of claim 1, wherein the diploid embryo is cloned or multiplied to make multiple copies of the embryo.
